(19) United States
(12) Patent Application Publication
(10) Pub. No.: US 2003/0070681 A1
(43) Pub. Date: Apr. 17, 2003

(54) Dosing system

(75) Inventor: Goran Rydgren, Bunkeflostrand (SE)

Correspondence Address: SCHIFF HARDIN & WAITE, 6600 SEARS TOWER, 233 S WACKER DR, CHICAGO, IL, 60606-6473, US

(73) Assignee: Siemens Elema AB

(21) Appl. No.: 10/243,822

(22) Filed: Sep. 13, 2002

(30) Foreign Application Priority Data
Oct. 4, 2001 (SE) 0103309-1

(51) Int. Cl. A61M 16/00

(52) U.S. Cl. 128/204.18

(57) ABSTRACT

A dosing system has a dosing unit for delivering an amount of an additive into an inspiration gas from a mechanical ventilator an introducer arrangement for introducing an indicator material into the inspiration gas at an onset time within an inspiration phase, and a control arrangement which monitors a level of a component of the expiration gas related to the indicator material during a successive expiration phase and to control the dosing unit to vary the timing of delivery of the additive dependent on both the monitored level and the onset time.

### BACKGROUND OF THE INVENTION

1. Field of the Invention

The present invention relates to a dosing system for supplying an additive to an inspiration gas, in particular to a dosing system in which the timing of the delivery of the dose within an inspiration phase is controlled by monitoring the contents of the expiration gas.

2. Description of the Prior Art

It is known that the additive nitrogen monoxide (NO) (also called nitric oxide) is preferentially taken up in the alveoli of the lung. It is also known that the amount of NO taken up by the lung can be varied by varying the timing (that is one or more of the start time, the end time, duration and presence within an inspiration phase) of delivery of a dose of NO into an inspiration gas which is to be supplied to a patient. Thus the operation of an NO dosing unit may be controlled dependent on the level of NO detected in expiration gas so as to improve the efficiency of the dosing.

Certain additives, such as surfactants and prostacyclin, because they are effective only if they reach a certain region of the lung, usually are dosed throughout an entire inspiration phase in an attempt to ensure that they reach that lung region. This is both inefficient and costly as almost 90% of the dosed additive is typically present in expiration gas.

### SUMMARY OF THE INVENTION

It is an object of the present invention to provide a dosing system for supplying an additive to an inspiration gas wherein the aforementioned problems associated with known dosing systems are avoided or minimized.

This object is achieved in accordance with a first embodiment of the present invention in a dosing system and a method for controlling timing of delivery of an amount of additive into an inspiration gas, wherein an indicator material is introduced into the inspiration gas at a known unset time within the inspiration phase, a level of a component of expiration gas related to the indicator material is monitored during a successive expiration phase, and the timing of the delivery of the additive is adjusted dependent on both the monitored level and the onset time.

Thus by monitoring the expiration gas for components related to the indicator, or example changes in level of the component consequent to different introduction times of the indicator, the timing of delivery of a dose of an additive into an inspiration gas may be adjusted. A reduced amount of additive may then be delivered into the respiratory system of the patient.

The indicator material is selected so as to be preferentially taken up within a known region of the respiratory system of the patient, most usefully within a region associated with a preferential action or take-up the additive. Thus the timing of the delivery of the dose may be varied to ensure a delivery of the additive to the known region.

The above object also is achieved in accordance with the principles of the present invention in an embodiment of the invention directed to a dosing monitor, having an arrangement for introducing an indicator material into an inspiration gas during an inspiration phase, the indicator material indicating take-up of an additive within the respiratory system of the patient, and having a detector which monitors a level of a component of the expiration gas related to the indicator material during a successive expiration phase, the detector providing an output indicative of this level.

### DESCRIPTION OF THE DRAWINGS

FIG. 1 is a schematic illustration of a dosing system according to the present invention operably connected to a ventilator.

FIG. 2 is a schematic illustration of a dosing monitor according to the present invention operably connected to a ventilator.

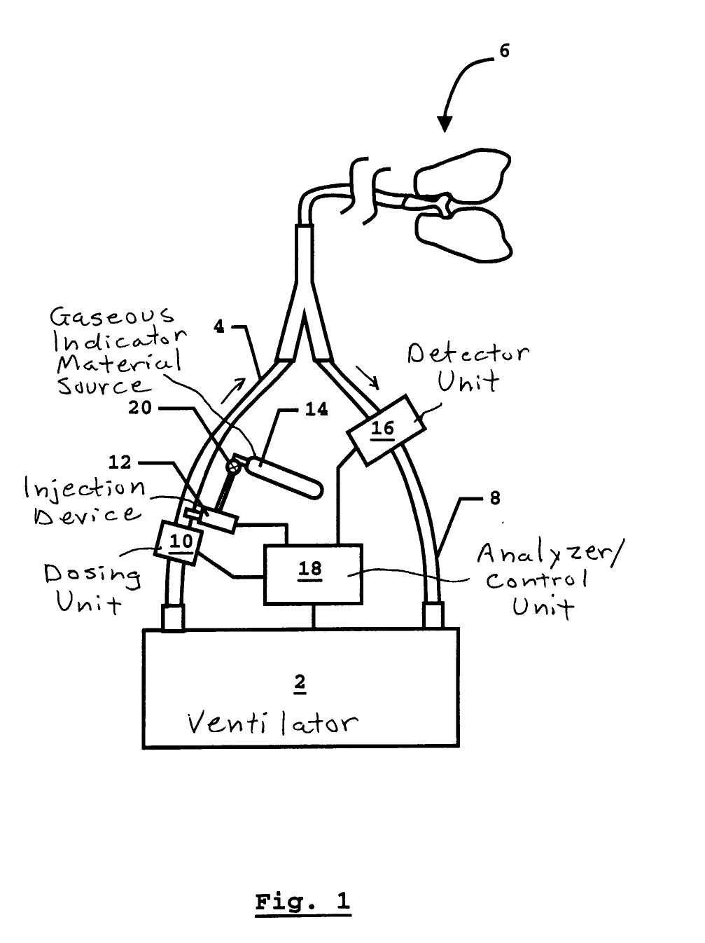

Considering now the example of a dosing system as shown in FIG. 1, a standard ventilator 2, of known operation and construction, is arranged to supply an inspiration gas through an inspiration line 4 and to the respiratory system of a patient 6 during inspiration phases of patient breathing cycles. The ventilator 2 also is arranged to receive, during expiration phases, expiration gas from the respiratory system 6 through an expiration line 8.

As shown in FIG. 1, the exemplary dosing system has a dosing unit 10, an injection device 12, a source of gaseous indicator material 14, a detector unit 16 and an analyzer/control unit 18. The dosing unit 10, such as a conventional nebulizer or vaporizer, is connected to the inspiration line 4 and is operable to dose inspiration gas within that line 4 with a predetermined amount of an additive, such as a physiologically active additive having a dilatory or narcotic effect. The operation of the dosing unit 10 to deliver the dose of additive is controlled by the analyzer/control unit 18 which is configured to control among other things, the start time, stop time or dosing profile (variations in additive flow with time during the delivery) of the dosing unit 10.

The analyzer/control unit 18 of the present embodiment also is operably connected to the ventilator 2 to receive a signal indicative of the initiation of an inspiration phase and thus the onset of supply of inspiration gas by the ventilator 2. It will be appreciated by those skilled in the art that a dedicated flow meter within the inspiration line 4 may alternatively be used to generate the indicative signal. This signal is employed by the analyzer/control unit 18, in a known manner, as a timing signal against which the operation of the dosing unit 10 is controlled. The analyzer/control unit 18 is additionally connected to control the injection device 12 to inject a known amount of indicator material into the inspiration gas flow at a variable onset time within the inspiration phase. This onset time may be readily established using a known mechanical or electronic timer initiated by the timing signal already used by the analyzer/control unit 18 in the control of the dosing unit 10.

The injection device 12, which may be a on/off valve or proportional valve of known construction, is used to introduce a known amount of marker indicator into inspiration gas, either together with or separate from the delivery of the additive, within the inspiration line 4. It will be appreciated by those skilled in the art that the dosing unit 10 itself also may be employed as the means to introduce the indicator material in accordance with the invention. The injection device 12 is connectable to the source of indicator material 14 through the operation of the flow control valve 20.

The source 14 in this embodiment is a commercially available cylinder of pressurized NO diluted in nitrogen to provide a known concentration of the NO. Other gaseous indicator material may be substituted for NO, such as carbon Monoxide (CO) which also is preferentially taken up in the alveoli of the respiratory system 6, or Nitrous Oxide (N₂O) which is preferentially taken up in the bronchiole of the respiratory system. A material which is transformed into a readily detectable gas when taken-up in the respiratory system 6, for example the enzyme L-arginine which is known to oxidize to produce NO as it penetrates cell membranes of the respiratory system 6, also may be employed as the indicator material.

The detector unit 16 is arranged to detect levels of a component of expiration gas passed from the respiratory system of the patient 6 which is related to the indicator material. As can be seen from the examples of indicator material provided above, dependent on the indicator material selected the component may be the indicator material itself or a substance which results from an interaction of the indicator material with the respiratory system 6. As illustrated in the embodiment of FIG. 1, the detector unit 16 may be conveniently connected to the expiration line 8 and is configured to provide an output indicative of the detected level to the analyzer/control unit 18.

In one mode of operation of the dosing system illustrated by FIG. 1 the dosing unit 10 is supplied with a medicament, such as a surfactant, which is effective only if taken up in the alveoli of the respiration system 6. The analyzer/control unit 18 is provided with operating instructions, such as in the form of program code portions when the analyzer/control unit 18 is microprocessor based, to control the injection device 12 to introduce NO at a different onset time in each of a number of inspiration phases and to store a value representing the monitored level of the component of expiration gas in expiration phases consequent to each of the inspiration phases, as obtained from the detection unit 16. The analyzer/control unit 18 then operates to analyze the stored values to determine an onset time at which the take up of NO in the respiratory system is greatest. Since, in this example, it is desired to ensure that the medicament is taken up in the alveoli then the analyzer/control unit 18 sets the start time for delivery of medicament by the dosing unit 10 to be the determined onset time for NO.

If the additive delivered by the dosing unit 10 is intended for take up in another region of the respiratory system 6, such as, for example, in the bronchial tree for asthma medicine, then using NO as the marker material and operating the system as described above to obtain the onset time for maximum NO take-up the analyser/control unit 18 may be arranged to set a start time for delivery time shifted (here delayed) by a predetermined amount dependent on the separation between the take-up region of the indicator material and the target delivery region of the additive. It will be appreciated that the analyzer/control unit 18 may be programmed with a number of pre-set time shifts which may be automatically selected dependent on user input indicator take-up and additive target regions of the respiratory system 6 (perhaps input as indicator material and additive). Alternatively, in this case the indicator source 14 may be replaced with an indicator which also is preferably taken-up in the bronchial tree, for example N₂O, a suitable detector provided in the detector unit 16, and the analyzer/control unit 18 configured to operate the dosing device 10 to start the delivery of the additive at a time equal to an onset time determined for greatest take-up of the indicator.

In a different mode of operation of the system according to FIG. 1, the dosing device 10 is operated by the analyzer/control unit 18 dependent on the efficacy of the medicament in changing the physical properties of the respiratory system 6, as indicated by the level of component in expiration gas monitored by the detector unit 16. In the present example the source of indicator 14 is selected to indicate a preferential take-up in a different part of the respiratory system 6 to that of the additive delivered by the dosing unit 10, and in particular the region of take-up indicated by the indicator material is after that of the additive in the direction of inspiration gas flow from the ventilator 2. To illustrate this mode of operation it is assumed that the additive is selected to provide bronchial spasm relaxation, such as a cortisone-based medicament like pulmicort™. The indicator gas in the source 14 is CO, or any other material which preferentially interacts with the alveoli, and is preferably introduced into inspiration gas within the inspiration line within the same inspiration phase as the additive is delivered by the dosing unit 10. The detector unit 16 is arranged to monitor levels of CO in expiration gas within the expiration line 8 and to provide an output to the analyzer/control unit 18 indicative of the same. The analyzer/control unit 18 is programmed to compare a difference between a level of CO introduced and a monitored level within the same breathing cycle with a reference level and to inhibit delivery of the additive in subsequent inspiration phases when the comparison indicates a take-up of the CO in the alveoli. This shows that the additive has been effective in removing the spasm and is no longer required. The analyzer/control unit 18 is further arranged to re-initiate delivery by the dosing unit 10 when the comparison indicates that take-up of CO is being reduced.

Additionally, the source 14 may include a second indicator material, such as N₂O, selected to interact preferentially with the bronchiole of the respiratory system 6, and the detector unit 16 configured to also monitor levels of this gas within the expiration gas. The analyzer/control unit 18 is configured to operate as described for the previous mode to determine an onset time at which the interaction of the indicator N₂O is greatest and further to track this determined time with the number of inspiration phases within which the additive is delivered. This provides information about the position and duration of the spasm region within the bronchiole and may be used to vary the start time of delivery of the additive to match the movement of the spasm through the bronchiole.

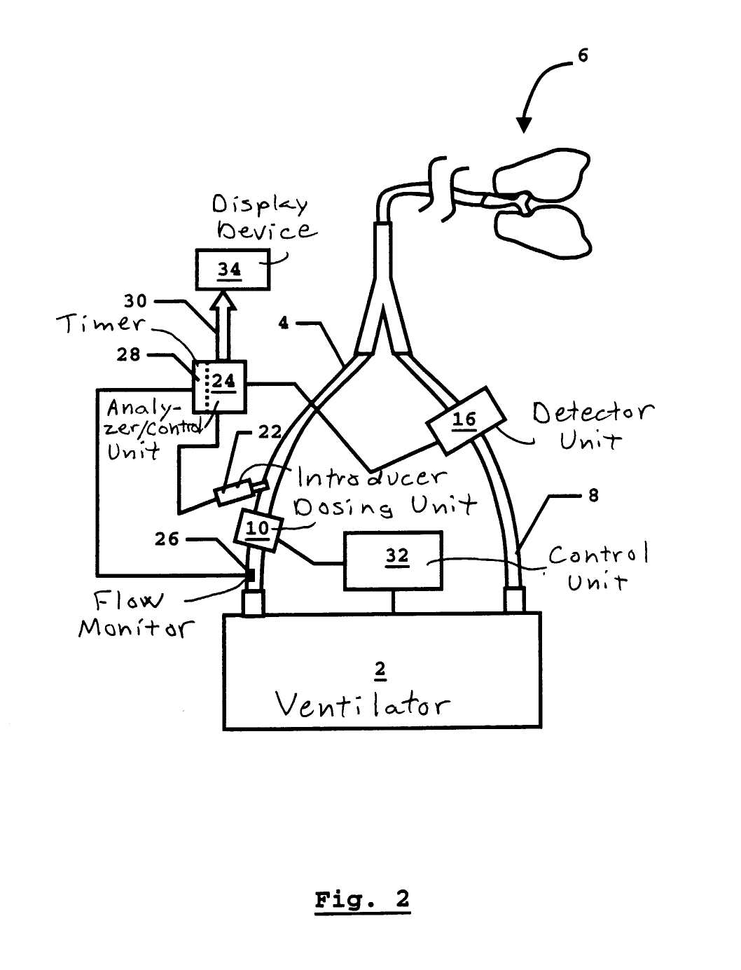

The dosing monitor according to the present invention which is illustrated in the exemplary embodiment of FIG. 2. In FIG. 2, items which are common to both the embodiments of FIG. 1 and FIG. 2 are provided with identical reference numerals. A dosing monitor has a an indicator material introducer 22, which may be the same as the injection device 12 of FIG. 1; a detector unit 16 for detecting components of expiration gas related to indicator material introduced into inspiration gas by the means 22; an analyzer/control unit 24 and a flow monitor 26. The analyzer/control unit 24 is arranged to receive a signal from the flow monitor 26 which is positioned, in use, in the inspiration line 4 connected to the ventilator 2, indicating a start of an inspiration phase. Such flow monitors are well known in the art of mechanical ventilation and may be configured to monitor one or both of flow rate and pressure within the inspiration line 4. It will be appreciated by those skilled in the art that the flow monitor 26 allows operation of the dosing monitor independent of the make and type of the ventilator 2.

The signal from the flow monitor 26 is employed by the analyzer/control unit 24 as a timing signal to start a timing device 28 which forms a part of the analyzer unit (in a similar manner to the use of timing signal from the ventilator 2 by the analyzer/control unit of the embodiment of FIG. 1). After a predetermined time has elapsed a signal from the timing device 28 causes the analyzer/control unit 24 to trigger the introducer 22 to introduce an amount of indicator material into the inspiration line 4. The detector unit 16 operates to provide an output to the analyzer/control unit 24 indicative of the monitored component level.

The analyzer/control unit 24 is configured to operate in a manner substantially similar to those described with respect to the various modes of operation of the analyzer/control unit 18 of FIG. 1 to provide an output signal 30 related to the dosing of inspiration gas within the inspiration line 4 by the dosing device 10 in a known manner, under control of a control unit 32. This output signal 30 may be provided by the analyzer/control unit 24 in a form suitable to drive a display device 34 to provide a visual indication of, for example, delivery timing parameters (such as start time, end time or duration) for input into the control unit 32 to vary the operation of the dosing device, or of the efficacy of the additive dosed by the dosing device 10.

Although modifications and changes may be suggested by those skilled in the art, it is the intention of the inventor to embody within the patent warranted hereon all changes and modifications as reasonably and properly come within the scope of his contribution to the art.

I claim as my invention:

1. A dosing system comprising:
a dosing unit for delivering an amount of additive into an inspiration gas adapted for delivery to a subject experiencing alternating inspiration phases and expiration phases;
an introducer for introducing an indicator material into said inspiration gas at an onset time within one of said inspiration phases; and
a control unit for monitoring, in expiration gas in an expiration phase following said one of said inspiration phases, a level of a component of said expiration gas related to said indicator material, and for controlling said dosing unit to vary a timing of delivery of said additive dependent on said level and said onset time.

2. A dosing system as claimed in claim 1 wherein said introducer introduces said indicator material into said inspiration gas at respective onset times which vary in different inspiration phases, and wherein said control unit controls said dosing unit to vary said timing of delivery of said additive dependent on variations in said level relative to variations in said onset time.

3. A dosing system as claimed in claim 2 wherein said control unit determines, from said variations in said level, an onset time for a maximum take-up of said additive by said respiratory system, and controls said dosing unit to start said delivery of said additive dependent on said onset time for maximum take-up.

4. A dosing system as claimed in claim 2 wherein said control unit determines, from said variations in said level, at least one of an upper onset time indicative of a first predetermined take-up of said additive by said respiratory system and a lower onset time indicative of a second predetermined level of take-up of said additive by said respiratory system, and controls said dosing unit to vary a duration of said delivery of said additive dependent on said at least one of said upper onset time and lower onset time.

5. A dosing system as claimed in claim 1 further comprising a source of said indicator material connected to said introducer, said source of said indicator material comprising a plurality of different gases, respectively preferentially taken up at different known regions of said respiratory system, as said introducer material, to said introducer.

6. A method for controlling timing of delivery of an additive into an inspiration gas supplied to a respirating subject experiencing alternating inspiration and expiration phases, said method comprising the steps of:
introducing an indicator material into said inspiration gas at a known onset time within one of said inspiration phases;
monitoring a level of a component related to said indicator material in expiration gas during an expiration phase following said one of said inspiration phases; and
adjusting the timing of said delivery of said additive dependent on said level and said onset time.

7. A method as claimed in claim 6 comprising:
varying said onset time in each of a plurality of inspiration phases;
monitoring said level of said component in each of a plurality of expiration phases respectively following said plurality of inspiration phases, and comparing the respective levels of said component in said plurality of expiration phases; and
adjusting said timing of delivery of said additive dependent on said comparison of said levels.

8. A method as claimed in claim 7 wherein the step of comparing said levels comprises determining an onset time indicating a maximum take-up of said additive by said respiratory system, and wherein the step of adjusting the timing of said delivery comprises varying a start of said delivery dependent on said onset time indicating a maximum take-up of said additive.

9. A dosing monitor comprising:
an introducer for introducing an indicator material, indicating take-up of an additive within a respiratory system of a patient, into inspiration gas during an inspiration phase experienced by said patient; and
a detector which monitors a level of a component of an expiration gas, in an expiration phase following said inspiration, related to said indicator material, and which generates an output dependent on said level.

10. A dosing monitor as claimed in claim 9 further comprising an analyzer which compares said output of said detector in each of a plurality of expiration phases, at least one of said plurality of expiration phases, following said inspiration phase, and for providing an output dependent on the comparison.
